(19) United States
(12) Patent Application Publication
(10) Pub. No.: US 2004/0208832 A1
(43) Pub. Date: Oct. 21, 2004

(54) Color-coded therapy unit

(76) Inventor: Michael Timothy Bates, Belle Mead, NJ (US)

Correspondence Address: ELI LILLY AND COMPANY, PATENT DIVISION, P.O. BOX 6288, INDIANAPOLIS, IN, 46206-6288, US

(21) Appl. No.: 10/493,191

(22) PCT Filed: Nov. 1, 2002
(86) PCT No.: PCT/US02/31610

Related U.S. Application Data
(60) Provisional application No. 60/335,225, filed on Nov. 2, 2001.

(51) Int. Cl. A61K 38/28; A61L 9/04; A61K 9/14

(52) U.S. Cl. 424/46; 514/3; 514/12

(57) ABSTRACT

Color-coded pulmonarily administrable therapy units (10) of medicine and a method for delivery. In one form of the invention, a pulmonarily administrable therapy unit (10) includes a disposable container (11) and a dry powder (16) held within the disposable container (11), which dry powder (16) includes a therapeutic agent. The disposable container (11) is insertable into an inhaler (30) and openable by operation of that inhaler (30) to allow therapeutic agent to escape from the disposable container (11) for inhalation. The disposable container (11) has a color-coding (26) that represents a total quantity of the therapeutic agent within the disposable container (11). The invention also encompasses a dosing system for pulmonary delivery via an inhaler (30) of a therapeutic agent, including first and second therapy units (10) separately loadable into an inhaler (30) and each holding a different quantity of a protein or peptide therapeutic agent. The first and second therapy units (11) are differently color-coded (26), which color-codings (26) represent the different total quantities of therapeutic agent of the units (11).

### BACKGROUND OF THE INVENTION

The present invention pertains to the pulmonary administration of protein and peptide pharmaceuticals to systemic circulation, and, in particular, to certain indicia associated with such pharmaceuticals.

Recently there has been much attention given to the pulmonary route of administration as a possible means for systemic delivery of protein and peptide pharmaceuticals. These medicines currently are delivered via injection. In order for the pulmonary route to be a viable alternative to injections, there must be an ability of the patient to accurately control his/her dosing.

In the field of injectable protein and peptide pharmaceuticals, human growth hormone, as an example, previously has been provided in disposable pen cartridges that are color-coded for use with similarly color-coded reusable injection pens. While the color-coding in this cartridge/pen system indicates the concentration of the multiple doses of medication contained in the cartridge, this system and its color-coding is of limited usefulness to the user. For one thing, the human growth hormone medicine still must be injected with a needle, which is undesirable for some users. Furthermore, the actual amount of medicine administered is not determinable by analyzing just the color-coding, but rather is a function of the volume that is dialed up on and then delivered by use of the pen. Thus, after a dose is administered, if a patient cannot recall the volume dialed up on the pen for delivery, simply knowing the concentration of the medicine that has been injected will not allow the person to determine with certainty the amount of therapeutic agent actually administered.

It would be desirable to provide a dosing system and method for the pulmonary administration of protein and peptide pharmaceuticals which assist a user in administering a suitable total dose of such pharmaceuticals.

### BRIEF SUMMARY OF THE INVENTION

The present invention provides pulmonarily administrable therapy units including a protein or peptide therapeutic agent, which therapy units are color-coded as to the amount of included therapeutic agent to thereby facilitate proper dosing by a user. Based on relatively simple instructions from a treating medical professional, such as to administer one or more therapy units colored, for example, blue, or to administer one or more therapy units colored, for example, blue, as well as one or more therapy units colored, for example, red, the user can operate an inhaler as needed with the therapy unit(s) to pulmonarily deliver a sufficient amount of medicine.

In one form thereof, the present invention provides a method for pulmonary administration of a protein or peptide therapeutic agent, comprising the steps of providing a color-coded therapy unit comprising the therapeutic agent and a container in which the therapeutic agent is held, wherein the color-coding represents a total quantity of the therapeutic agent within the container, loading the color-coded therapy unit into an inhaler, causing the container of the therapy unit loaded in the inhaler to open to allow therapeutic agent to escape from the container, and inhaling from the inhaler therapeutic agent that escapes from the opened container.

In another form thereof, the present invention provides a pulmonarily administrable therapy unit for use with an inhaler, comprising a disposable container insertable into the inhaler, a dry powder held within the disposable container and which comprises a therapeutic agent, the disposable container openable by operation of the inhaler to allow therapeutic agent to escape from the disposable container for inhalation, and wherein the disposable container comprises a color-coding that represents a total quantity of the therapeutic agent within the disposable container.

In another form thereof, the present invention provides a dosing system for pulmonary delivery via an inhaler of a therapeutic agent, comprising a first therapy unit and a second therapy unit. The first therapy unit comprises a first quantity of a protein or peptide therapeutic agent and a container in which the first quantity of therapeutic agent is held. The first therapy unit is loadable into the inhaler in which the first therapy unit container is openable to allow escape of therapeutic agent for pulmonary delivery by the inhaler. The first therapy unit comprises a color-coding that represents the first quantity. The second therapy unit comprises a second quantity of the protein or peptide therapeutic agent and a container in which the second quantity of therapeutic agent is held. The second therapy unit is loadable into the inhaler in which the second therapy unit container is openable to allow escape of therapeutic agent for pulmonary delivery by the inhaler. The second therapy unit comprises a color-coding that represents the second quantity and which is different from the color-coding of the first therapy unit.

One advantage of the present invention is that a method and dosing system for the pulmonary administration of protein and peptide pharmaceuticals is provided which use a color-coded therapy unit to assist the user in administering a proper quantity of such medication.

Another advantage of the present invention is that a color-coded pulmonarily administrable therapy unit may be provided which, both before and after its use, provides a visual indication to a user as to the amount of protein or peptide therapeutic agent originally present.

Still another advantage of the present invention is that a color-coded pulmonarily administrable therapy unit may be provided which permits a user to visually observe whether or not the contained protein or peptide therapeutic agent has been released therefrom and into the inhaler with which the unit is used.

Yet another advantage of the present invention is that a pulmonarily administrable therapy unit and its associated packaging may each be provided with a color-coding associated with the quantity of the inhaleable protein or peptide medication.

Yet another advantage of the inventive method and dosing system is that it is simple to understand and use.

### BRIEF DESCRIPTION OF THE DRAWINGS

The above-mentioned and other advantages and objects of this invention, and the manner of attaining them, will become more apparent, and the invention itself will be better understood by reference to the following description of embodiments of the invention taking in conjunction with the accompanying drawings, wherein:

FIG. 1 is a diagrammatic perspective view of one form of color-coded therapy unit suitable for use with a pulmonary delivery device;

FIG. 2 is a diagrammatic front view of a blister pack for the therapy unit of FIG. 1;

FIG. 3 is a diagrammatic front view of a pulmonary delivery device loaded with the therapy unit of FIG. 1; and

FIG. 4 is a diagrammatic front view in partial cross-section of the pulmonary delivery device of FIG. 3 being operated to puncture the therapy unit container to permit escape of its held dry powder.

Corresponding reference characters indicate corresponding parts throughout the several views. Although the drawings represent an embodiment of the present invention, the drawings are not necessarily to scale, and certain features may be exaggerated or omitted in some of the drawings in order to better illustrate and explain the present invention.

### DETAILED DESCRIPTION OF THE INVENTION

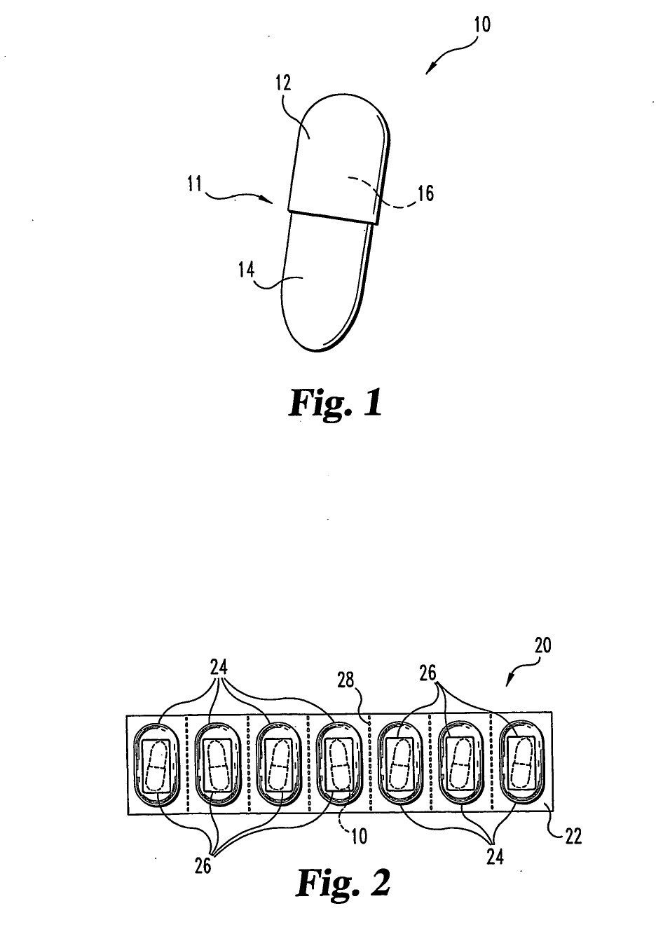

Referring now to FIG. 1, there is shown an exemplary embodiment of a pulmonarily administrable therapy unit of the present invention. The therapy unit, generally designated 10, is provided in a capsule form in which is contained a protein or peptide therapeutic agent in a form suitable for pulmonary administration.

Therapy unit 10 includes a capsule-shaped container 11 constructed from mating shell halves 12 and 14 in a known configuration, and an inhaleable dry powder 16 filled within container 11. Container 11 is adapted to be openable by the type of pulmonary delivery device or inhaler in which it is loaded for use. In a preferred embodiment, container shell halves 12 and 14 are made of a material, such as a gelatinous or thin plastic material, which is translucent or transparent, and further which is puncturable or sliceable open by one or more piercing or cutting elements of an associated inhaler to allow dry powder 16 to escape from the container. Capsule container 11 would also serve its purpose if the inhaler in which it were loaded for use pulled apart the shell halves 12 and 14 for release of its dry powder contents. Alternate containers that permit the escape of medicine contained therein are also within the scope of the present invention. For example, the container may be a blister pack or pouch, such as made of puncturable foil, in which the inhaleable dry powder is held in loose format, or in other words the pack contains no capsule in which the dry powder is further contained.

Dry powder 16 includes the protein or peptide therapeutic agent or active ingredient, but optionally may include other materials as well, such as carriers, bulking agents or excipients. Exemplary therapeutic agents for different therapy units include insulin, human growth hormone, glucagon-like peptide or GLP, and analogs, fragments and derivatives of each. The total amount of therapeutic agent within dry powder 16 is controlled by the manufacturer as each therapy unit 10 serves as an individual dose of medication at the time of its use. The term individual dose herein refers to the fact that each therapy unit is typically to be separately loaded and its medicine inhaled by use in an inhaler, and not that every user will find one individual dose to be enough, as depending on the needs of a given user, to administer the proper amount of medicine at a given time, multiple individual therapy units may be required to be administered by reloading the inhaler and repeating its use as appropriate to deliver the medicine required.

Therapy unit 10 is color-coded to provide a user with information as to the total quantity of therapeutic agent contained in container 11. For example, in a preferred embodiment, in the case of human growth hormone (hGH), therapy units are provided in a multitude of different strengths, such as four, five, six or seven, with each strength therapy unit being color-coded differently from the other strengths. Similarly, in the case of insulin, color-coding may be used to indicate that the therapy unit is one of two or three different strengths. Still other numbers of available therapy unit strengths may be accommodated by the color-coding within the scope of the invention.

With respect to insulin, it is expected that the strength will represent the actual mass of that therapeutic contained within that unit. For example, a 0.9 mg strength pulmonary insulin capsule contains 0.9 mg of insulin powder. Taking into account known losses in the administration of dry powder insulin to the deep lung, such a 0.9 mg strength capsule may correspond or be equivalent to a two international unit (IU) subcutaneous injection of a standard solution or suspension of the same. Similarly, a 2.7 mg strength capsule and a 4.5 mg strength capsule may respectively correspond to a six IU and ten IU subcutaneous injection of a standard solution or suspension of the same.

While the therapy unit color-coding does represent to the users and treating medical professionals a total quantity of therapeutic agent within a given therapy unit, which total quantity in the case of insulin is provided in the form of a dry powder mass-based strength, the total quantity may be identified differently than the above-identified strength numbers. For example, and subject to regulatory approval, such capsules may have their total quantity of insulin additionally or alternatively labeled in terms of the subcutaneous injection equivalents, such as the two, six or ten IU amounts described above.

The color-coding of therapy unit 10 can be achieved in a variety of ways within the scope of the invention. For example, for the shown two-part construction of capsule container 11, one of the capsule shell halves, such as shell half 12, could in its entirety be of a particular identifying color, with the other capsule shell half 14 being an alternate color, such as a neutral color, or even colorless and clear. Alternatively, only a portion of one or both of the shell halves 12 and 14, or possibly all of both shell halves 12 and 14, could be provided with the identifying color. Although a color-coding using a single particular color to identify the total quantity of the therapeutic agent is preferred for simplicity, combinations of two or more colors may be employed within the scope of the invention.

In the preferred embodiment, the color-coding of the shell half or halves, as the case may be, is not opaque so as to preclude recognition of the existence of dry powder therein. Instead, the color-coding is preferably provided by a tinting of the translucent or transparent container material, resulting in the interior contents of the container visible therethrough appearing to be the color signifying the dose quantity. Alternatively, such a see-through color-coding may be provided as a transparent or translucent, colored coating of the translucent or transparent container 10. The ability of a person to visually observe whether or not dry powder is contained within a given container aids a user in determining whether inhalation of a dose has occurred.

In an embodiment of the present invention when the therapeutic agent is human growth hormone (hGH) available in five different strength capsules, and with reference to the parts labeled in the exemplary therapy unit of FIG. 1, a first strength dose is held in a container 11 which appears a first color due to a see-through tinting of transparent shell halves 12 and 14 in their entirety, a second and greater strength dose is held in a different container 11 which appears a second color due to a see-through tinting of transparent shell halves 12 and 14 in their entirety, and similarly three additional, greater and different strength doses are held in different containers 11 which respectively appears a third color, a fourth color and a fifth color due to a see-through tinting of their respective transparent shell halves 12 and 14 in their entirety.

The capsule form therapy unit 10 is preferably provided to a user in a protective packaging that is color-coded identically to that therapy unit. As shown in FIG. 2, a set of seven therapy units 10 are provided in a blister pack which but for its color-coding is of known design. As is conventional, blister pack 20 includes a relatively rigid base 22 with seven cavity-defining blisters 24 that are each filled with an individual capsule therapy unit 10. Capsule filled cavities 24 are sealed by a more readily puncturable material, such as an aluminized foil, which a therapy unit 10 is passed or punched through for use in an inhaler when its respective blister 24 is pressed on in a conventional fashion. For such blister packaging, the exterior of the blisters 24 are colored as shown at 26 with the same color-coding as their respective contained therapy units 10. Although not shown, the outer surface areas of foil through which the therapy units are punched out are also colored-coded the same as their respective contained therapy units 10. In an embodiment where part of the blister pack is clear such that the coloring of the therapy unit is visible, no additional coloring 26 is required on that part.

Although the pack is shown including seven therapy units, such as if the pack held a one-week supply of one-a-day doses, the number of doses within the pack may be varied by the manufacturer. In addition, scoring 28 arranged vertically in FIG. 2 permits each pack to be divided if the user so desires. Blister pack 20 may hold capsules all filled with the same quantity of medicine, in which case all of the color-codings 26 are the same color(s). Alternatively, pack 20 may hold capsules filled with different quantities of medicine, in which case the color-codings will be different to appropriately indicate such different quantities.

The color-coding of blister pack 20 provides the user with information about the therapy unit hidden within the packaging. The color-coding of blister pack 20 as well as the capsule therapy units 10 contained therein permits a user to double check the amount of medicine in the unit to be administered, once while the therapy unit is hidden in the packaging and then a second time when being loaded into an inhaler. Still further, if therapy unit 10 is taken out and loaded into the inhaler, and the blister pack 20 is discarded, a user can determine the amount of medicine by analyzing the color-coding of the loaded therapy unit.

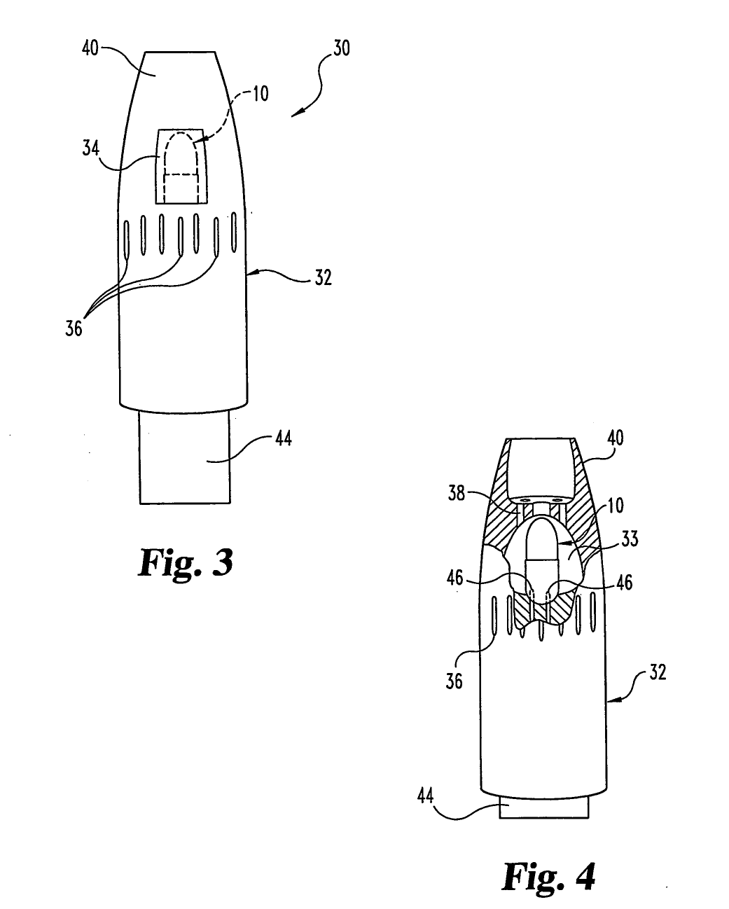

Referring now to FIGS. 3 and 4, there is diagrammatically shown an inhaler, generally designated 30, that is operable to deliver a dry powder medication into a user in a known fashion. The inhaler description herein is illustrative and not intended to be limiting, as the design of the inhaler, other than its ability to be loaded with and administer an individual pulmonarily administrable therapy unit 10, is not material to the present invention, and types of inhalers other than that shown may be substituted for inhaler 30 within the scope of the invention.

Other than its viewing window 34 described below, inhaler 30 is of a known design and is therefore only described briefly and in general terms herein. Inhaler 30 includes a main body 32 that defines an internal chamber 33 into which a single therapy unit 10 is loadable by a user. Therapy unit 10 is so loaded by, for example, disassembling a component part, such as an inhalation section, of main body 32 to access the chamber 33, manually inserting the therapy unit, and then reassembling that component part to main body 32. Viewing window 34 consists of a clear plastic insert covering an opening in main body 32 which allows the therapy unit 10 loaded in the inhaler to be visible. Clear refers to the window being colorless or sufficiently untinted such that the color-coding of the therapy unit is ascertainable to a user's naked eye. Although only a portion of therapy unit 10 is shown in FIG. 3, in the preferred embodiment the viewing window is sufficiently large to allow the entire capsule to be visible.

A series of longitudinally extending slits 36 arranged around the main body 32 permit air to be drawn into chamber 33 during inhalation. Openings 38 at the top of chamber 33 port to a duct within an inhalation section 40 that forms the top end of main body 32.

Movably mountable to inhaler main body 32 is a piercing assembly, which is shown in a retracted position in FIG. 3 and an operational position in FIG. 4. The piercing assembly includes a longitudinally extending plunger 44 that is spring biased to the position shown in FIG. 3. When a user manually presses plunger 44 to drive it into main body 32 against the force of the biasing spring, a piercing element including a pair of pins 46 is shifted upward within compartment 33. When plunger 44 has been fully shifted by a user and has reached the position shown in FIG. 4, pins 46 have penetrated or passed through container shell half 12. When plunger 44 is released by a user and is spring biased back to the position shown in FIG. 3, pins 46 are retracted out of the therapy unit 10, which permits dry powder 16 to escape by gravity from the puncture holes formed in container 11.

Further details as to the design and operation of the type of inhaler shown at 30 are disclosed in U.S. Pat. Nos. 4,069,819 and 4,995,385, the entire disclosures of which are incorporated herein by reference.

The method of using therapy unit 10 and inhaler 30 will be even further understood in view of the following additional explanation. When a user desires to administer medicine, the proper therapy unit can be selected based on its color-coding. For example, the user may have been instructed by her treating physician to take a blue colored therapy unit. If that blue colored therapy unit 10 is housed within blister pack 20, in a standard fashion a user punches it from the pack through the foil seal. The user then loads the punched out blue colored therapy unit 10 into inhaler chamber 33 by manipulating the inhaler 30 as appropriate, ultimately resulting in the loaded inhaler being arranged as shown in FIG. 3. Viewing window 34 allows a user to confirm the loaded therapy unit 10, such that even if the user postpones administration until a later time at which a question may arise as to whether a, or what size, therapy unit is loaded, that user need not disassemble the inhaler to determine what is loaded therein.

In preparation for administrating the medicine, inhaler 30 is then mechanically operated to open container 11 to access the contained dry powder 16. Specifically, while maintaining inhaler 30 upright, plunger 44 is plunged by the user to cause pins 46 to penetrate container 11. When the user then releases plunger 44 to allow the piercing assembly to automatically return to its retracted state, the withdrawal of pins 46 from container 11 allows the dry powder 16 to escape therefrom through the newly created holes.

The inhaler is then moved such that inhalation section 40 is inserted into, for example, the user's mouth. When the user proceeds to inhale or suck air through inhaler 30, dry powder already within chamber 33 due to it dropping thereto by gravity from punctured container 11, along with dry powder drawn from container 11 through the pin created holes by an inhalation-created suction effect, is drawn into the user for delivery to her deep lungs (for systemic delivery) within the air stream that passes through slits 36, into chamber 33, and through openings 38 and the duct formed by inhalation section 40.

After inhaling, the user may again observe therapy unit 10 through viewing window 34 to visually determine that an administration has occurred. This determination involves verifying that the dry powder 16 has been emptied at least in part, and preferably completely or at least substantially so, from container 11. While the container puncturing accesses all of the contained dry powder or therapeutic agent, the amount of therapeutic agent that during administration has emptied from the container 11, and which then actually reached the user's lungs to enter the bloodstream, may be different than for clinical subjects due to various factors, such as the inhaling force that can be applied by that user. The treating physician, when instructing a given user as to which dose or doses (such as the blue therapy unit described above) to administer, is to have taken into account the efficiency of the pulmonary delivery for that user.

When the user is satisfied that the dry powder and therefore the therapeutic agent has been inhaled, the inhaler can be manipulated to remove the punctured and now at least partially emptied container 11 from chamber 33. This step naturally can be postponed until another medicine administering use of the inhaler is required. Until removed, the container 11 can be viewed through viewing window 34 to allow a user to ascertain, by considering the container color-coding, information about the last inhaled dose. After container removal, if another therapy unit 10 is required to be delivered to achieve administration of the proper amount of medicine, the method can be repeated immediately. For example, the instructions to the user from the treating physician may have been to administer with the inhaler either one or more additional blue therapy unit(s), or one or more red or green therapy unit(s), or a combination of the two, after the first blue colored therapy unit has been administered. Otherwise, the inhaler can be reassembled until subsequently needed, at which time the method can be repeated.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

I claim:

1. A method for pulmonary administration of a protein or peptide therapeutic agent, comprising the steps of:
providing a color-coded therapy unit comprising the therapeutic agent and a container in which the therapeutic agent is held, wherein the color-coding represents a total quantity of the therapeutic agent within the container;
loading the color-coded therapy unit into an inhaler;
causing the container of the therapy unit loaded in the inhaler to open to allow therapeutic agent to escape from the container; and
inhaling from the inhaler therapeutic agent that escapes from the opened container.

2. The method of claim 1 wherein the color-coding of the therapy unit comprises a color-coding of at least a portion of the container.

3. The method of claim 2 wherein the color-coded container is translucent or transparent to permit observation of the therapeutic agent within the container through the color-coding.

4. The method of claim 1 wherein the therapeutic agent comprises human growth hormone.

5. The method of claim 4 wherein the color-coding of the therapy unit consists of a single color.

6. The method of claim 1 wherein the therapeutic agent comprises insulin.

7. The method of claim 6 wherein the color-coding of the therapy unit consists of a single color.

8. The method of claim 1 wherein the therapeutic agent comprises a glucagon-like peptide.

9. The method of claim 1 wherein the therapeutic agent comprises a dry powder.

10. The method of claim 1 further comprising the step of providing a viewing window in the inhaler to permit visual observation of the color of the therapy unit loaded in the inhaler.

11. The method of claim 1 wherein the step of causing the container of the therapy unit to open comprises operating the inhaler to move a puncturing member to pierce at least one hole in the container.

12. The method of claim 1 further comprising the step of removing the opened container from the inhaler after the inhaling step, and loading a second therapy unit into the inhaler, the second therapy unit comprising a container in which is held the therapeutic agent in a total quantity different from the total quantity held in the previously loaded therapy unit prior to its opening, and wherein the second therapy unit comprises a color-coding that represents the total quantity of the therapeutic agent within the second therapy unit container and which is different from the color-coding of the previously loaded therapy unit.

13. The method of claim 1 further comprising the step of providing the color-coded therapy unit within a blister pack which is color-coded similarly to the therapy unit.

14. A pulmonarily administrable therapy unit for use with an inhaler, comprising:
a disposable container insertable into the inhaler;
a dry powder held within said disposable container, said dry powder comprising a therapeutic agent;
said disposable container openable by operation of the inhaler to allow therapeutic agent to escape from the disposable container for inhalation; and
wherein said disposable container comprises a color-coding that represents a total quantity of said therapeutic agent within said disposable container.

15. The pulmonarily administrable therapy unit of claim 14 wherein said therapeutic agent comprises insulin.

16. The pulmonarily administrable therapy unit of claim 15 wherein said color-coding consists of a single color.

17. The pulmonarily administrable therapy unit of claim 14 wherein said color-coded container is translucent or transparent to permit observation of said dry powder within said container through the color-coding.

18. The pulmonarily administrable therapy unit of claim 14 wherein said therapeutic agent comprises human growth hormone.

19. A dosing system for pulmonary delivery via an inhaler of a therapeutic agent, comprising:
a first therapy unit comprising a first quantity of a protein or peptide therapeutic agent and a container in which said first quantity of therapeutic agent is held, said first therapy unit loadable into the inhaler in which said first therapy unit container is openable to allow escape of therapeutic agent for pulmonary delivery by the inhaler, said first therapy unit comprising a color-coding that represents said first quantity; and
a second therapy unit comprising a second quantity of the protein or peptide therapeutic agent and a container in which said second quantity of therapeutic agent is held, said second therapy unit loadable into the inhaler in which said second therapy unit container is openable to allow escape of therapeutic agent for pulmonary delivery by the inhaler, said second therapy unit comprising a color-coding that represents said second quantity and which is different from the color-coding of said first therapy unit.

20. The dosing system of claim 19 wherein said therapeutic agent comprises insulin.

21. The dosing system of claim 19 wherein said therapeutic agent comprises human growth hormone.

22. The dosing system of claim 19 wherein said color-coding of each of said first therapy unit and said second therapy unit consists of a single color.
